(19) United States
(12) Patent Application Publication
(10) Pub. No.: US 2007/0135924 A1
(43) Pub. Date: Jun. 14, 2007

(54) Joint replacement prosthesis, joint replacement mounting stud and method

(76) Inventor: Alex R. Verhoogen, Spokane, WA (US)

Correspondence Address: WELLS ST. JOHN P.S., 601 W. FIRST AVENUE, SUITE 1300, SPOKANE, WA, 99201, US

(21) Appl. No.: 11/482,261

(22) Filed: Jul. 7, 2006

Related U.S. Application Data
(60) Provisional application No. 60/750,846, filed on Dec. 14, 2005.

(51) Int. Cl. A61F 2/30

(52) U.S. Cl. 623/18.11; 623/20.15

(57) ABSTRACT

A prosthesis anchor component is provided. The prosthesis anchor component has a mounting stud having an elongated body with a tubular distal end and a proximal end. The tubular distal end includes a tubular wall portion with an open leading end and a plurality of apertures extending through the tubular wall portion. The proximal end has a mounting interface for securing a joint interface onto the stud. A method is also provided.

### RELATED PATENT DATA

This application claims priority from U.S. Provisional Patent Application Ser. No. 60/750,846, which was filed Dec. 14, 2005, and which is incorporated by reference herein.

### TECHNICAL FIELD

The present invention pertains to medical devices and methods. More particularly, the present invention relates to prosthetic joint components and anchors for prosthetic joint components, and methods.

### BACKGROUND OF THE INVENTION

Joint arthroplasty involves the replacement of a natural joint with a prosthetic joint. Numerous joints can be replaced with prosthetic joints, including knee joints, hip joints and ankle joints. Such joints can be surgically replaced with a partial or complete prosthetic joint. For example, in the case of a knee joint a tibial component and a femoral component are installed onto a tibia and femur, respectively. In many cases, a tibial component has a tapered rectangular stem that is installed into a complementary receiving bore that is cut into cancellous bone in the tibia. Frequently, the stem is inserted into the bore along with bone cement. However, there is a need for further improvements when performing bone fixation of prosthetic joint implants.

Several joint prostheses incorporate the use of a stem which basically serves to prevent toggle or rotational displacement of the prosthesis. Such stems are found on a tibial base plate for total knee replacements, as well as on a resurfacing prosthesis for a femoral head as well as a humeral head. They are also found on a femoral component of unicondylar knee replacements. These stems have little fixation value, meaning they really do not offer significant strength to the attachment of the prosthesis to the bone. However, these stems do significantly compromise the ability to remove such prostheses, thus complicating the process of revision surgery. There is a need to provide a stem that better anchors the prosthesis to the underlying bone, while still being relatively easy to insert, and also relatively easy to remove for revision surgery without necessitating damage to the surrounding bone.

When inserting a knee prosthesis, the insertion of a tibial component causes the most soft tissue damage. The need to sublux the tibia forward on the femur and to insert the tibial component vertically, while avoiding damage to the femoral condyles and clearing the soft tissues medially, laterally, and anteriorly, greatly stresses soft tissues, occasionally even leading to the avulsion of a portion, or all, of the patellar tendon. The tightness of the patellar tendon will not infrequently cause a mild malrotation of the tibial component. This portion of the procedure is what prevents truly minimally invasive techniques.

FIG. 1 illustrates a femur 12 and a tibia 14 of a human knee joint 10 in a standing position. Soft tissue has been omitted in order to facilitate viewing of complementary condyles 16 and 18 on femur 12 and tibia 14, respectively.

FIG. 2 illustrates the components of FIG. 1 with knee joint 10 articulated to a bent, or sitting position. As shown by condyle 16 and condyle 18, the pair of condyles on each of femur 12 and tibia 14 are articulated into a new position.

FIG. 3 illustrates femur 12 and tibia 14 of knee joint 10 after preparation for receiving tibial and femur joint components (not shown). More particularly, both condyles (for example, condyle 16) are removed from femur 12 with a bone saw to provide cut surfaces 20, 22, 24, and 26. Likewise, both condyles (such as condyle 18) are removed from tibia 14 with a bone saw to provide cut surface 28. The cut surfaces on the tibia and femur are provided for receiving tibial and femur joint components.

FIG. 4 shows installation of a prior art tibial joint component 72 atop a tibia 14 of a knee joint 10 in which a rectangular bore or recess has been cut for receiving a tapered, rectangular stem on component 32. A femur joint component (not shown) is also mounted onto an end of femur 12. A set tool 30 is used to insert and drive component 32 into engagement with tibia 14. Tool 30 has an end configured to mate in complementary relation with a condyle interface component 34 atop a tibial base plate 33 in order to drive component 34 into tibia 14. However, in order to provide clearance for component 34 and tool 30, tibia 14 needs to be subluxed relative to femur 12. The resulting forward displacement of tibia 14 relative to femur 12 makes the procedure relatively invasive, especially relative to soft tissue movement and patella manipulation.

Accordingly, improvements are needed in order to reduce the need to sublux a tibia relative to a femur when installing a tibia joint interface component.

### SUMMARY OF THE INVENTION

A prosthesis and a prosthesis anchor component are provided with a mounting stud that has a perforated, or fenestrated, distal end that has an open leading end and a hollow interior portion configured to receive a vascularized pedicle of bone. A mounting interface is provided on a proximal end of the mounting stud that mates with a complementary mounting stud on a joint interface component. The mounting stud is installed into a receptacle within a bone adjacent a resected joint interface.

According to one aspect, a prosthesis anchor component has a mounting stud with an elongated body having a tubular distal end and a proximal end. The tubular distal end includes a tubular wall portion with an open leading end and a plurality of apertures extending through the tubular wall portion. The proximal end has a mounting interface for securing a joint interface onto the stud.

According to another aspect, a prosthesis anchor is provided with a mounting stem having a tubular leading end portion and a trailing end portion. The leading end portion has an open end communicating with a hollow interior portion and the trailing end portion has a mounting interface for securing a joint interface component onto the stud.

According to yet another aspect, a prosthesis is providing having a mounting anchor and a joint interface. The mounting anchor has a tubular leading end portion and a trailing end portion. The leading end portion has an open end communicating with a hollow interior portion and the trailing end portion has a first mounting interface. The joint interface component has a second mounting interface configured to mate in engagement with the first mounting interface to secure the joint interface component onto the mounting anchor.

According to even another aspect, a method is provided for affixing a joint interface component to a bone. The method includes: providing a mounting anchor having a fenestrated, tubular leading end portion and a trailing end portion with a female mounting interface and a joint interface component having a male mounting interface configured to mate with the first mounting interface; preparing a receptacle within a bone proximate a resected bone joint interface having a tubular groove extending about a vascularized pedicle of bone; inserting the mounting anchor into the receptacle with the tubular leading end received within the tubular groove; mating together the male mounting interface and the female mounting interface to engage together the mounting anchor and the joint interface.

### BRIEF DESCRIPTION OF THE DRAWINGS

Preferred embodiments of the invention are described below with reference to the following accompanying drawings.

FIG. 1 is a simplified lateral elevational view of a knee joint illustrating the tibia and femur in a standing position.

FIG. 2 is a simplified lateral elevational view of the knee joint of FIG. 1 with the tibia and femur in a flexed position.

FIG. 3 is a simplified lateral elevational view of the knee joint of FIG. 2 showing the tibia and femur after preparation for receiving tibial and femur joint components, respectively.

FIG. 4 is a simplified view of the knee joint of FIG. 3 showing a prior art technique for installing a prior art prosthetic knee joint wherein the tibia is subluxed, or translated in a frontal direction relative to the femur in order to provide installation space for the tibial implant component.

FIG. 5 is a simplified view of a knee joint being prepared to receive a prosthetic knee joint of the present invention showing articulation of the knee joint to accommodate preparation of the receptacle for receiving a stem for anchoring a tibial joint component.

FIG. 6 is a simplified partial breakaway view of the knee joint of FIG. 5 after preparation of the receptacle and showing insertion of a mounting stud for a tibial joint component into a receptacle in a tibia.

FIG. 7 is a simplified partial breakaway view of the knee joint of FIGS. 5-6 and showing mating of a joint interface component with the inserted mounting stud.

FIG. 8 is a simplified partial breakaway view of the knee joint of FIG. 7 showing the joint interface component affixed to the mounting stud.

FIG. 9 is a simplified perspective view of a knee joint prosthesis prior to assembling together the mounting stud and the knee joint interface.

FIG. 10 is a simplified partial breakaway frontal sectional view of the tibial knee joint component of FIGS. 7-9.

FIG. 11 is a simplified perspective view of a tibia (omitting the femur) during preparation of the receptacle using a guide tool and a cutting tool.

FIG. 12 is a simplified perspective view of a tibia (omitting the femur) during preparation of the receptacle using an alternative guide tool, drill bit, and hole saw.

FIG. 13 is a simplified perspective view of an alternative construction knee joint prosthesis prior to assembling together the mounting stud and the knee joint interface.

FIG. 14 is a simplified perspective view of a second alternative construction knee joint prosthesis prior to assembling together the mounting stud and the knee joint interface.

FIG. 15 is a partial breakaway view of a hip joint prosthesis mounted onto a femoral head according to a third alternative construction.

FIG. 16 is a simplified partial breakaway frontal sectional view of a fourth alternative construction tibial knee joint component.

FIG. 17 is a simplified partial breakaway frontal sectional view of a fifth alternative construction tibial knee joint component.

FIG. 18 is a simplified partial breakaway frontal sectional view of a sixth alternative construction tibial knee joint component.

FIG. 19 is an enlarged sub-assembly view, prior to assembly, of a tibial baseplate and anchor from the tibial knee joint component of FIG. 18.

FIG. 20 is a partial end view taken from below of the tibial baseplate of FIG. 19.

FIG. 21 is an enlarged sub-assembly view of the tibial baseplate and anchor connection taken from the encircled region 21 of FIG. 18.

FIG. 22 is a simplified partial vertical sectional view of a seventh alternative construction tibial knee joint component.

FIG. 23 is a sectional view of the tibial baseplate and anchor connection taken along line 23-23 of FIG. 22.

FIG. 24 is a simplified perspective view of an eighth alternative construction knee joint prosthesis prior to assembling together the mounting stud and the knee joint interface.

FIG. 25 is a simplified partial vertical sectional view of the tibial baseplate and anchor components of FIG. 24.

FIG. 26 is a sectional view of the tibial baseplate and anchor connection taken along line 26-26 of FIG. 25.

FIG. 27 is a simplified, exploded perspective view of a ninth alternative construction tibial knee joint component.

FIG. 28 is a simplified partial vertical sectional view of the tibial baseplate and anchor components of FIG. 27.

FIG. 29 is a sectional view of the tibial baseplate and anchor connection assembled together and taken along line 29-29 of FIG. 28.

### DETAILED DESCRIPTION OF THE PREFERRED EMBODIMENT

This disclosure of the invention is submitted in furtherance of the constitutional purposes of the U.S. patent Laws “to promote the progress of science and useful arts” (Article 1, Section 8).

Reference will now be made to preferred embodiments of Applicant's invention. Exemplary implementations are described below and depicted with reference to the drawings comprising joint interface components and mounting studs for joint interface components of joint prostheses.

While the invention is described by way of preferred embodiments, it is understood that the description is not intended to limit the invention to these embodiments, but is intended to cover alternatives, equivalents, and modifications which may be broader than the embodiments, such as is defined within the scope of the appended claims.

In an effort to prevent obscuring the invention at hand, only details germane to implementing the invention will be described in great detail, with presently understood peripheral details being incorporated by reference, as needed, as being presently understood in the art.

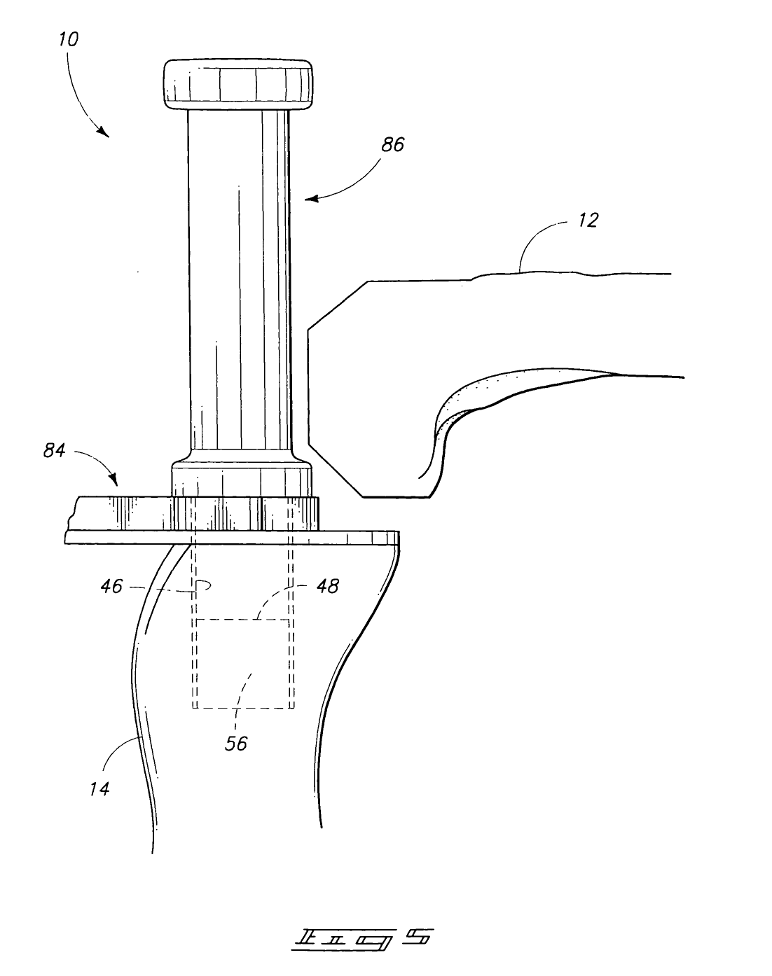

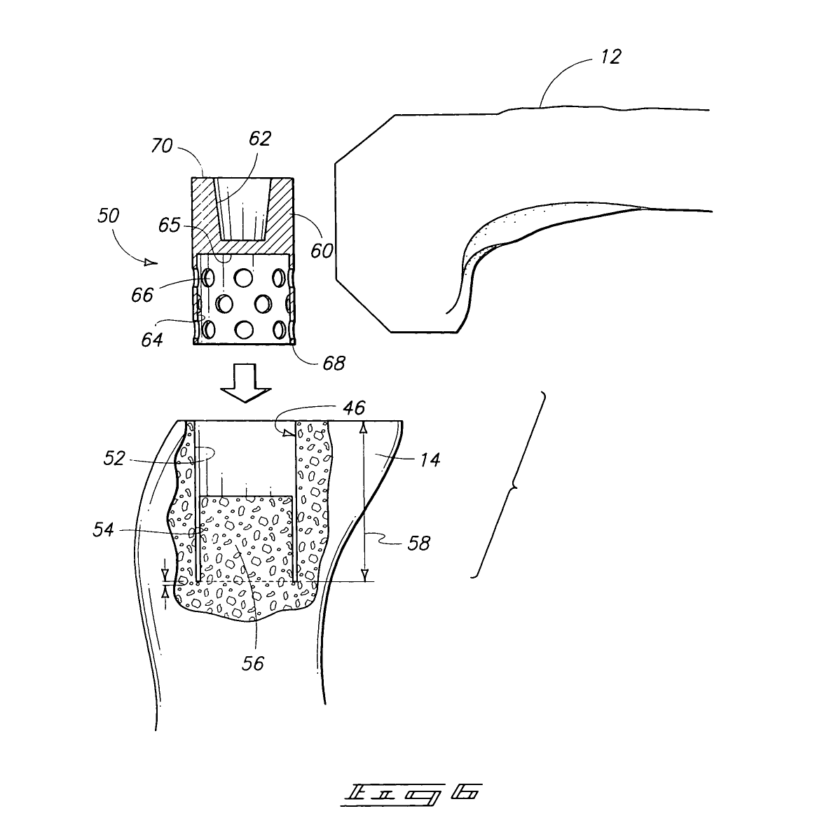

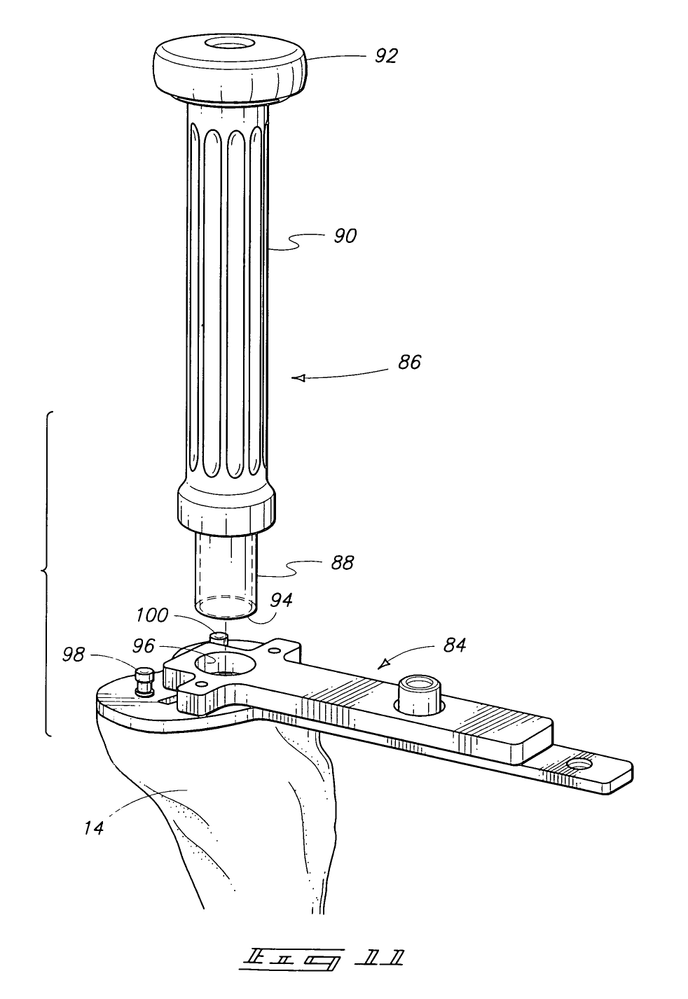

FIG. 5 illustrates one tool suitable for preparing a knee joint 10 to receive a mounting stud, or anchor 50 (see FIG. 6), according to the present invention. More particularly, a cutting tool 86 with a cylindrical cutting head is used to cut a cylindrical stem receptacle 46 within cancellous bone in tibia 14 of knee joint 10. According to one procedure, a drill bit, similar to bit 102 (of FIG. 12) is used to cut out a cylinder of bone down to depth 48 with a drill. Afterwards, cutting tool 86 is used to further cut a cylindrical groove, or kerf about a center core, or pedicle, 56 of vascularized bone. More particularly, a cutting head of cutting tool 86 is inserted into the cylindrical bore formed by the drill bit, after which a hammer is used to drive cutting tool 86 into the cancellous bone in order to create the cylindrical groove. The cylindrical cutting head is guided through a complementary cylindrical bore in a guide tool 84 that is affixed onto a top surface of tibia 14 using a pair of fasteners. The cylindrical cutting head has a length sufficient to cut a cylindrical groove with a depth that substantially matches a length of anchor, or stem 50 (see FIG. 6). In one case, the depth of the cylindrical groove is several millimeters less than the length of stem 50. Such depth ensures that a tibial joint interface component can subsequently be forcibly affixed onto the stem 50 after it has been forcibly driven into the resulting stem receptacle 46. If the receptacle is too deep, it might prove difficult to force the joint interface component into engagement with the stem.

FIG. 6 shows the formed stem receptacle 46 comprising cylindrical bore 52 and cylindrical groove, or kerf, 54 within tibia 14. Center core 56 is formed by bore 52 and kerf 54. Kerf 54 has a depth 58 that is a couple of millimeters shorter than the length of stem 50, according to one implementation. According to another implementation, depth 58 matches the length of stem 50. Stem 50 is inserted into receptacle 46.

Stem 50 comprises a unitary body 60 with a mounting interface 62 comprising a recess for receiving a Morse taper that has a frustoconical surface provided in a trailing end 70 of stem 50. A distal end of stem 50 has a cylindrical bore 64 extending from an open leading end 68 and terminating at an inner end wall 65. A plurality of holes, or fenestrations, 66 are provided in a cylindrical leading end of stem 50 for encouraging bone in-growth and through-growth from tibia 12 to stem 50.

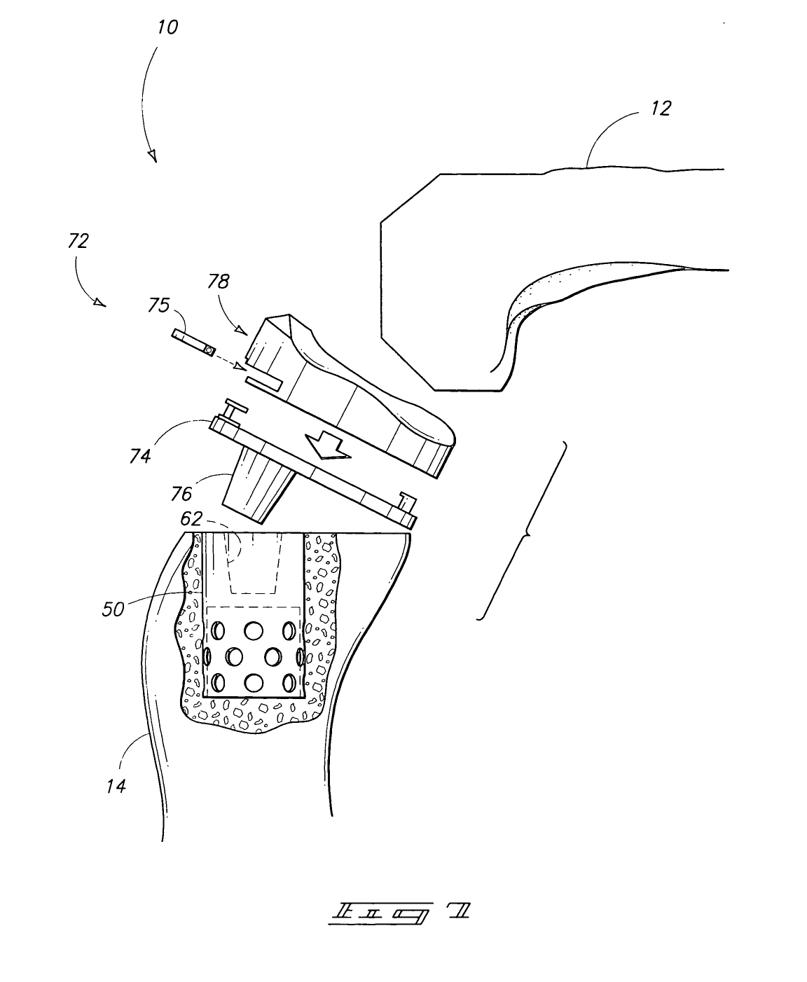

FIG. 7 illustrates a technique for installing joint interface component 72 onto stem 50 after stem 50 has been installed into a receptacle within tibia 14. Morse taper pin 76 is integrally formed onto tibial base plate 74. More particularly, pin 76 is received into complementary mounting interface 62 of stem 50 where they are tapped together to form a connection. Since plate 74 is relatively thin, pin 76 can be inserted into interface 62 without subluxing tibia 14 relative to femur 12, as is done according to prior art techniques. Subsequent to installing plate 74 onto stem 50, condyle interface component 78 is slid between clips on plate 74, according to techniques already known in the art, and a clip 75 is used to lock component 78 on top of plate 74.

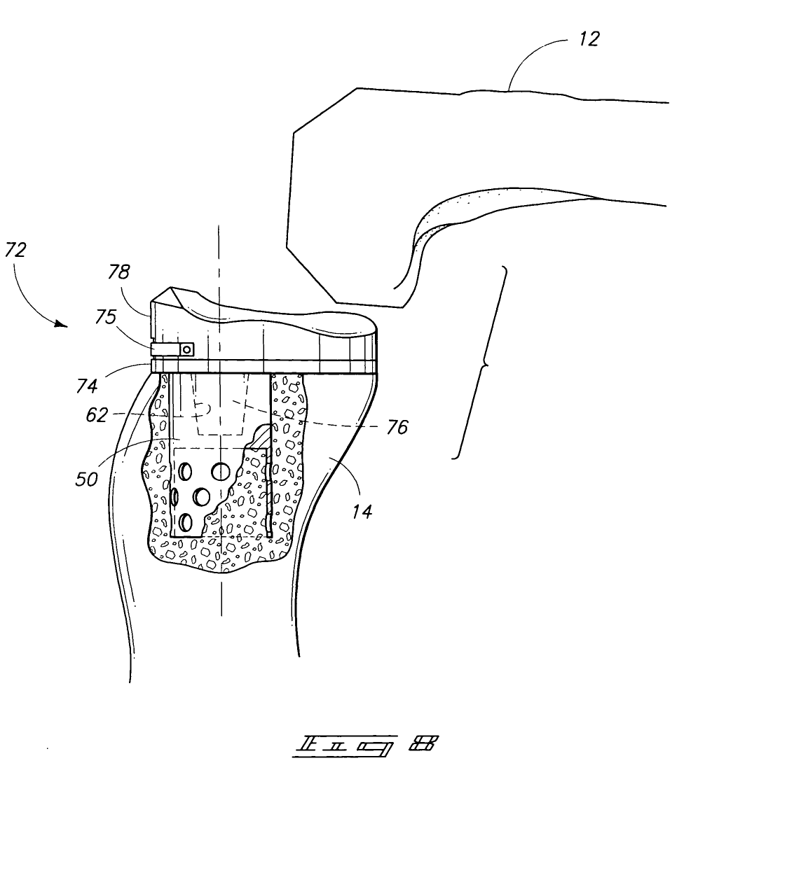

FIG. 8 shows joint interface component 72 installed onto tibia 14. Clip 75 is shown installed onto the front clips of plate 74, thereby locking component 78 on top of plate 74.

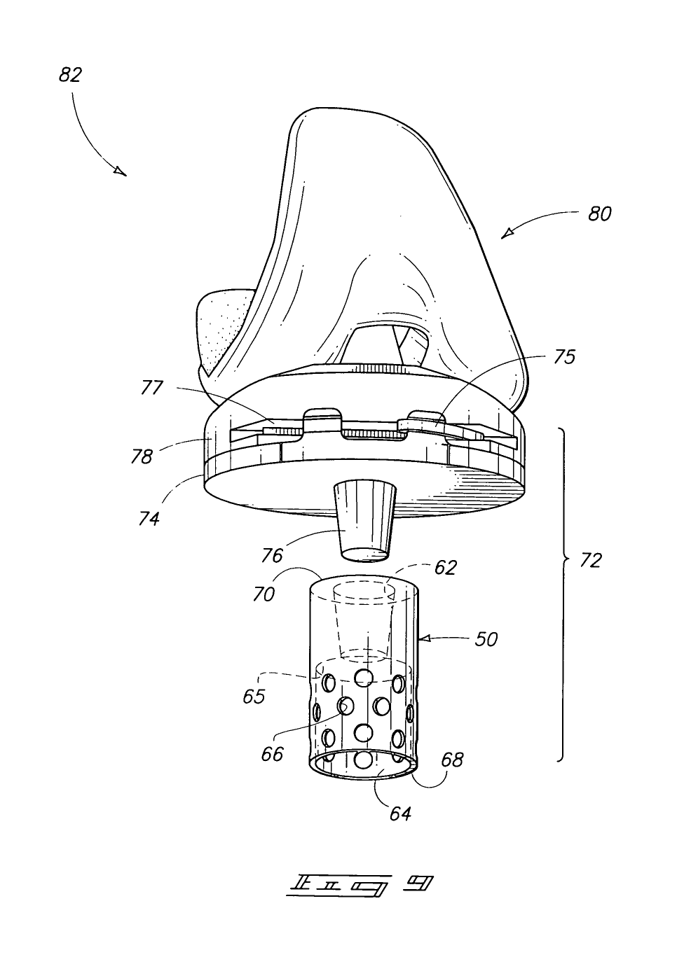

FIG. 9 illustrates a prosthesis 82 that includes a femur joint interface component 80 and a tibia joint interface component 72. Clip 75 is shown inserted into a slot 77 of component 78 and about one of the clips on base plate 74. Morse tapering pin 76 is shown integrally formed from plate 74. Furthermore, stem 50 is shown aligned for attachment with pin 76 by tapping together pin 76 into complementary frustoconical interface 62 of stem 50. According to one construction, base plate 74, pin 76, and stem 50 are constructed from a biocompatible material, such as a surgical stainless steel, or other compatible implant material.

FIG. 10 further illustrates components 72 and 80 of prosthesis 82 mounted onto tibia 14 and femur 12, respectively, of a knee joint 10. Stem 50 is shown with pin 76 locked within stem 50. Hence, stem 50 retains base plate 74 and component 78 onto tibia 14. As bone grows through the holes in stem 50, component 72 is locked onto tibia 14.

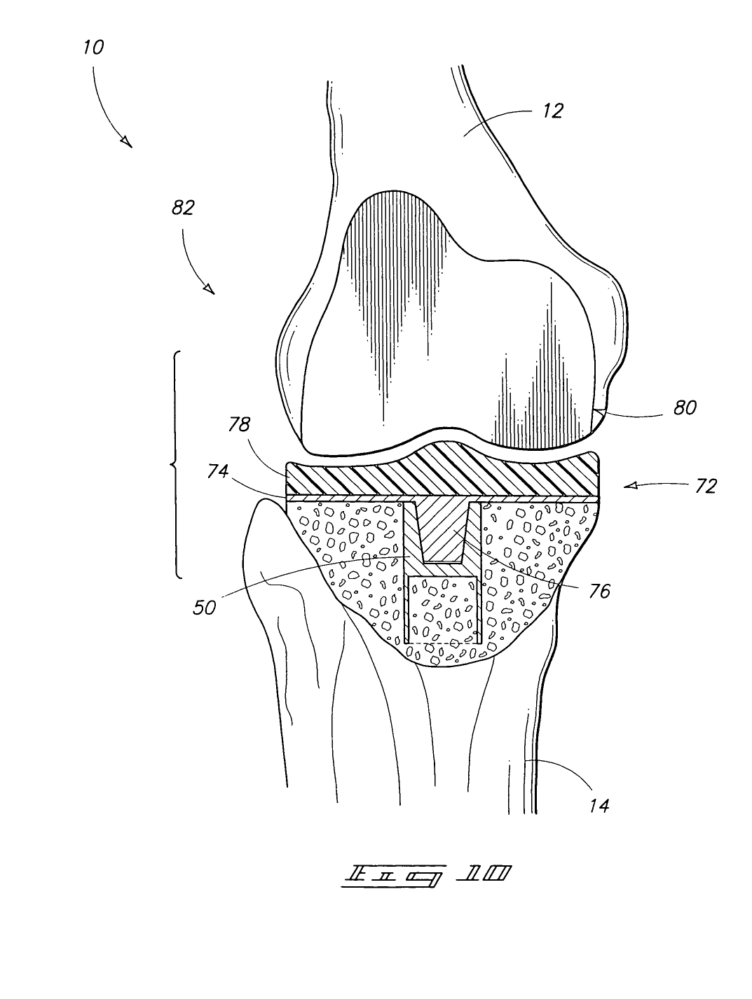

FIG. 11 further illustrates use of cutting tool 86 and guide tool 84 when cutting a cylindrical groove into the top of tibia 14, after cutting a bore into the top of tibia 14 with a drill bit. Tool 86 had a cylindrical cutting head 88, a shaft 90, and a head 92 that is configured to be hit with a drive hammer. A cylindrical cutting edge 94 on head 88 is received through cylindrical guide aperture 96 in guide tool 84. A pair of pins 98 and 100 are driven through apertures in tool 84 and into tibia 14 to fix positioning of guide aperture 96 in a desired location in order to accurately place the stem receptacle within tibia 14. Tool 86 and guide tool 84 are similar to tools presently being used to broach the proximal tibia for the stem, whether it is a cruciate, round, or square stem. However, the present tool 84 uses a cylindrical hollow core cutter head, instead of a square cutter head that is presently used. Tool 84 is inserted to a desired depth, and carefully removed, leaving the central core of bone. The resulting pedicle of bone remains vascularized through its base. The cutting slurry is left in the cut.

FIG. 12 shows an alternative configuration for preparing a stem receptacle within tibia 14. Guide tool 84 is mounted onto tibia 14 via pins 98 and 100 to accurately place guide aperture 96 atop tibia 14. Drill bit 102 is first guided through aperture 96 to cut a bore into tibia 14. A collar 106 is secured onto bit 102 to set a desired depth of cut using a threaded bore 110 and a set screw 108. A cylindrical bore in collar 106 is sized to receive bit 102, as well as cylindrical hole saw 104. After cutting a bore into tibia 14, hole saw 104 receives collar 106 at a desired cutting depth to form the cylindrical groove in tibia 14. When using hole saw 104, the cutting slurry is left in the cut.

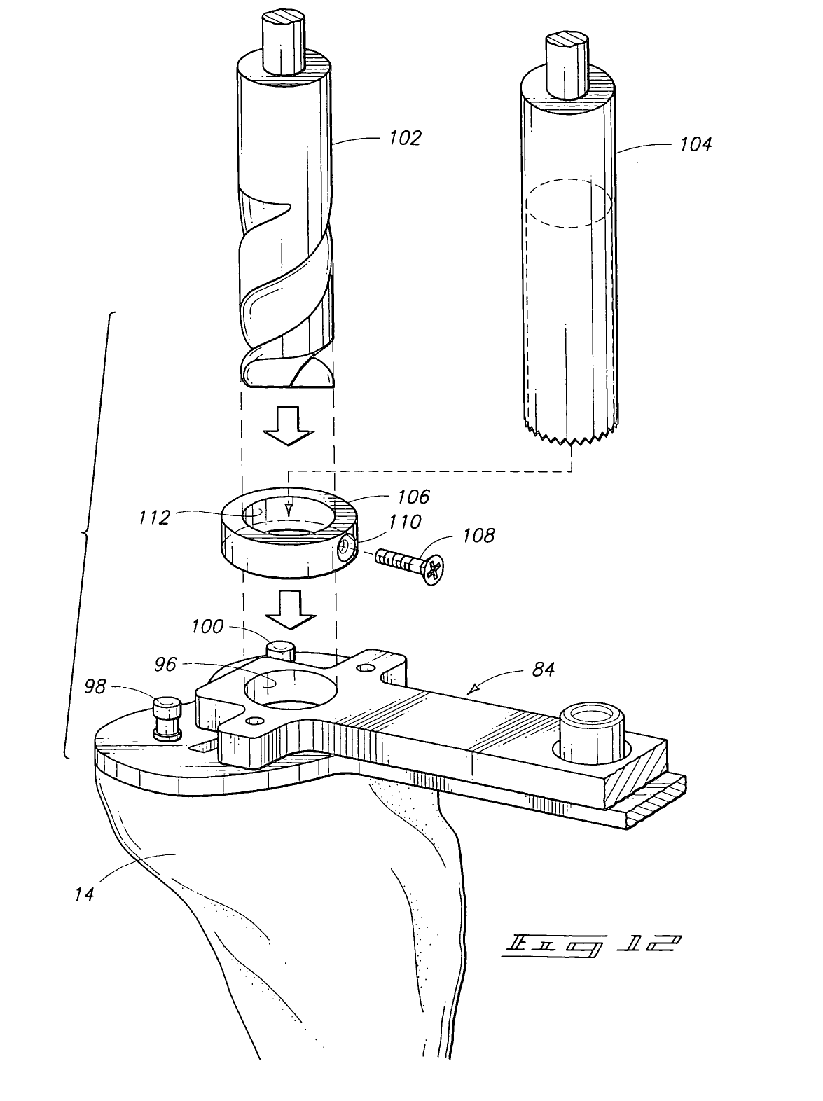

FIG. 13 illustrates an alternative construction prosthesis 182 comprising a femur joint interface component 80 and a tibia joint interface component 172. Prosthesis 182 is similar to prosthesis 82 (of FIG. 9). However, stem 150 has a rectangular, or square cross-sectional configuration. A proximal end of stem 150 has a frustoconical interface 162. A distal end 168 of stem 150 has a rectangular, tubular end portion with a plurality of holes, or fenestrations, 166. An inner bore 164 has a square cross-section that terminates in a square end wall 165. In one case, stem 150 is installed such that two sides extend in a fore-aft direction when installed in a tibia.

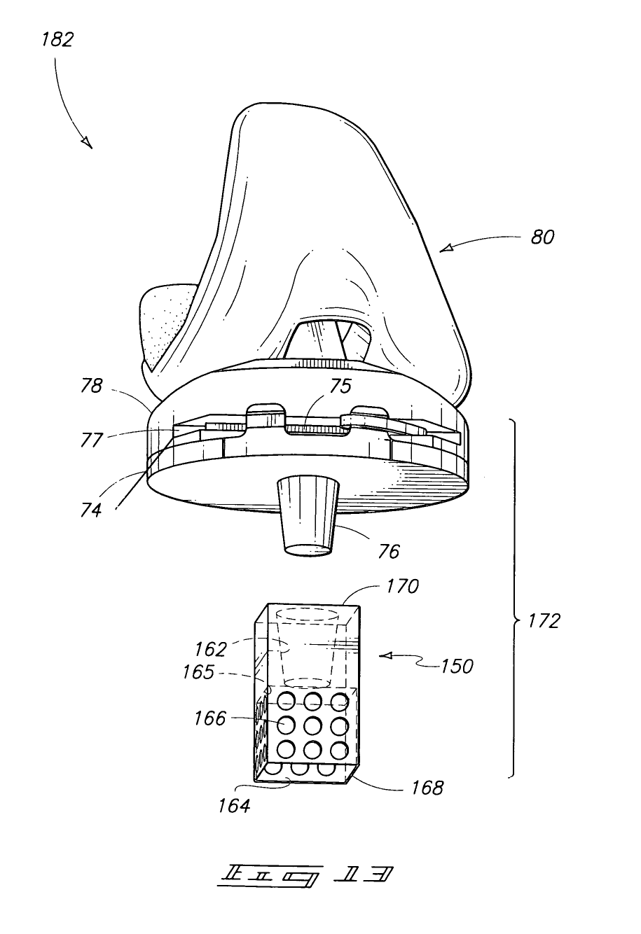

FIG. 14 shows a second alternative construction for a prosthesis 282 comprising a femur interface component and a tibia interface component 272. A stem 250 is cylindrical, but a plurality of slots 284 are provided in the proximal end 270 of stem 250. A pair of ribs 280 and 282 are formed between base plate 74 and pin 76, and are sized to be received within a selected, aligned pair of slots 284 in stem 250. Interface 262, end wall 265, bores 266, cylindrical bore 264, and cylindrical leading end 268 are similar to respective components on prosthesis 82 of FIG. 9.

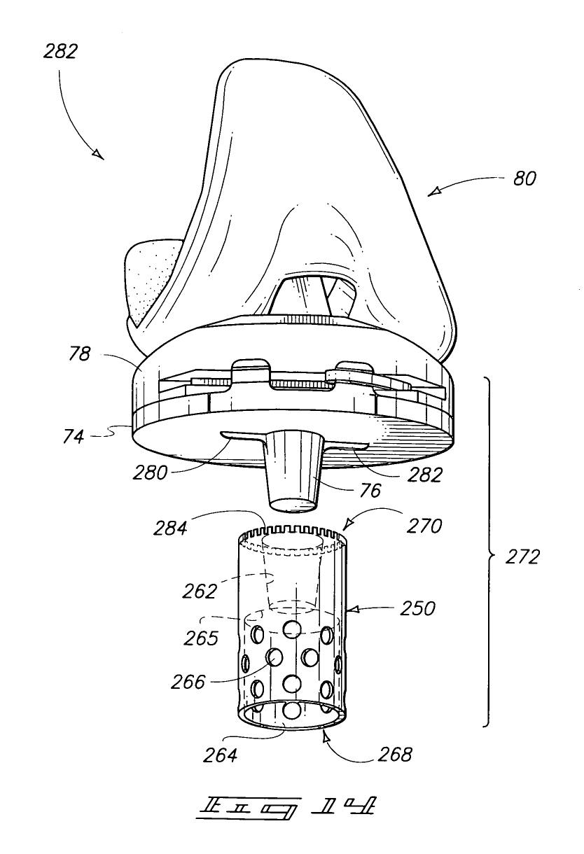

FIG. 15 illustrates a third alternative construction for a prosthesis for mounting a femoral head interface component 372 onto a femur 314. Component 372 includes a stem 350 and a joint interface component 380. Stem 350 is similar to stem 50.

According to the present techniques, a metal fenestrated hollow cylinder, open distally, with a complementary frustoconical interface, or female Morse taper proximally, is placed over a bone pedicle. The bone will grow through the fenestrations to secure the cylinder. The slurry, a mixture of bone particles and blood left in the cut, will facilitate this ingrowth.

The tibial base plate, with a male Morse tapering pin, is brought in from anteriorly and rotated into place around the patellar tendon. This avoids undue stress on the patellar tendon, and also avoids damage to the femoral condyles posteriorly. The rotation is aligned perfectly, and the Morse tapering pin is impacted into the Morse tapering recess. Removal of this prosthesis would involve disengaging the Morse tapering pin from the recess, and then simply cutting a cylinder over the anchor stem with a very slightly larger cylindrical cutter or hole saw.

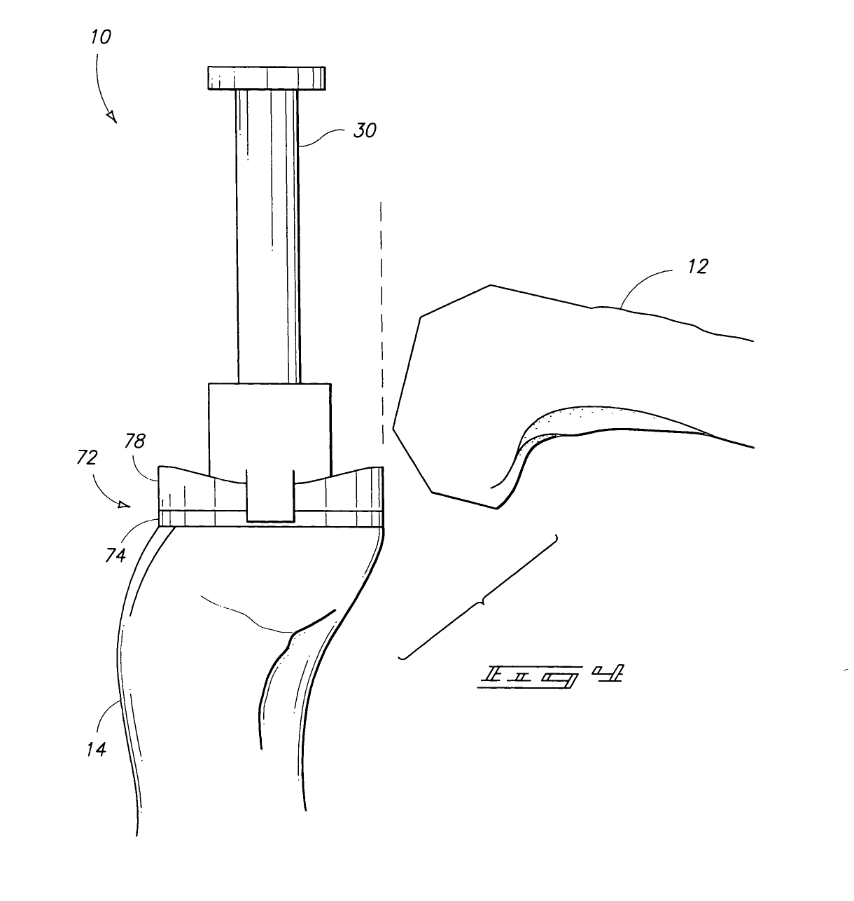

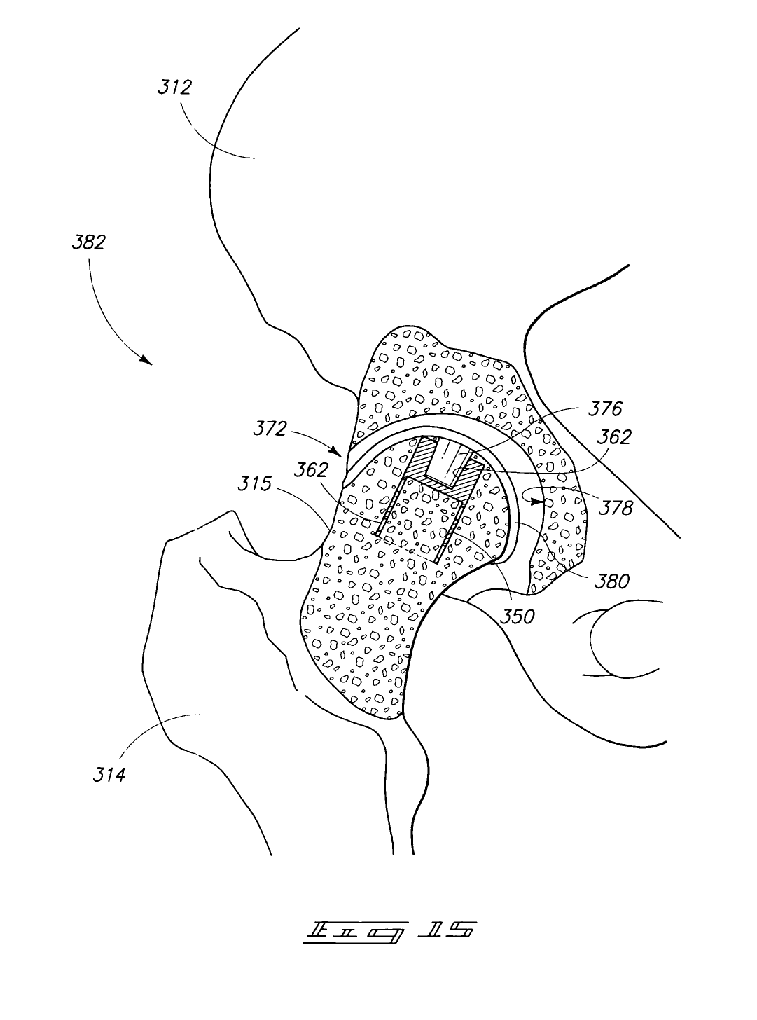

FIG. 16 illustrates a fourth alternative construction for a knee joint prosthesis 482 having a femoral joint interface component 80 and an alternatively constructed tibial joint interface component 472. Component 80 is provided on the end of a femur 12, whereas component 472 is provided on the end of a tibia 14. As shown, tibial joint interface component 472 comprises a condyle interface component 478 that is received atop a tibial baseplate 474 using a technique similar to affixing component 78 atop baseplate 74 (as shown in the embodiment depicted in FIG. 4). However, a Morse tapering pin 476 depends integrally from baseplate 474 to be received within a complementary frustoconical interface, or Morse tapering recess 462 of an anchor, or stem 450 that is received within tibia 14 using the techniques discussed above with respect to the previous embodiments.

Morse tapering pin 476 comprises a frustoconical pin that is forcibly received into a frustoconical Morse tapering recess 462 provided in anchor 450. Additionally, a threaded fastener 484 is received through a complementary bore 488 provided within Morse tapering pin 476. Fastener 484 is threaded into a female complementary threaded bore provided within anchor 450 in order to secure together Morse tapering pin 476 and Morse tapering recess 462. Optionally, a rib can be provided on one of Morse tapering pin 476 and Morse tapering recess 462, while a complementary engagement slot is provided in Morse tapering recess 462 (similar to ribs 280, 282 and slots 284 shown in the embodiment depicted in FIG. 14).

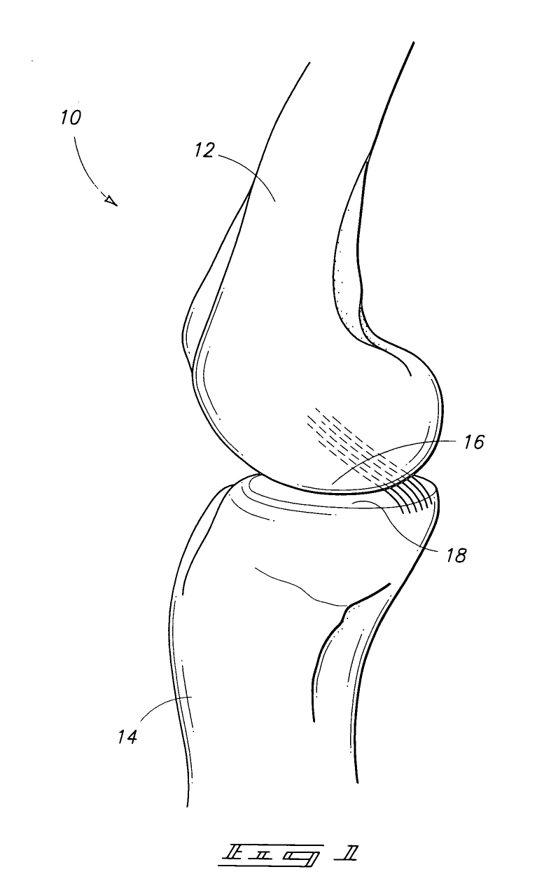

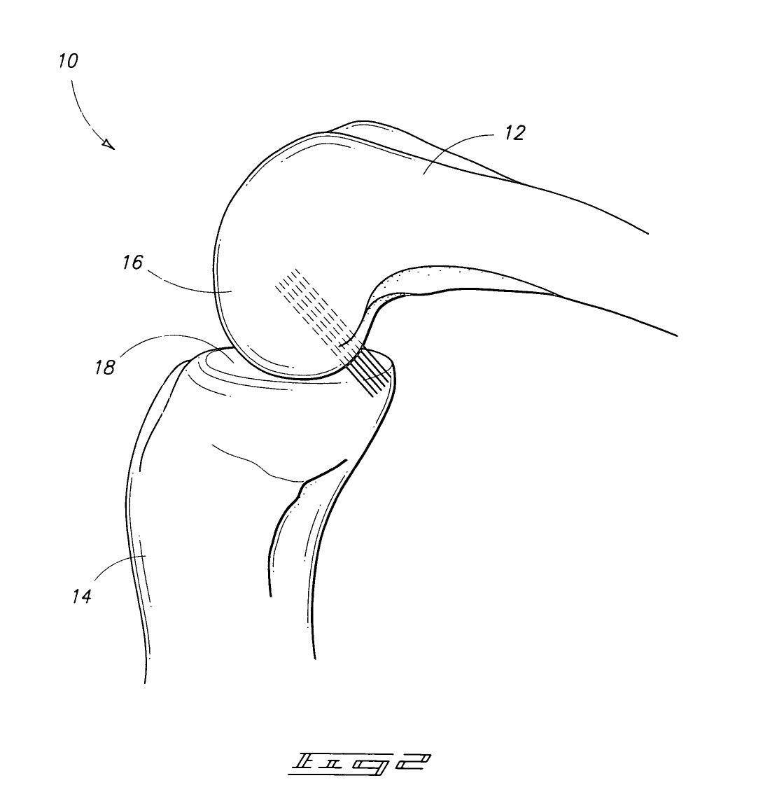

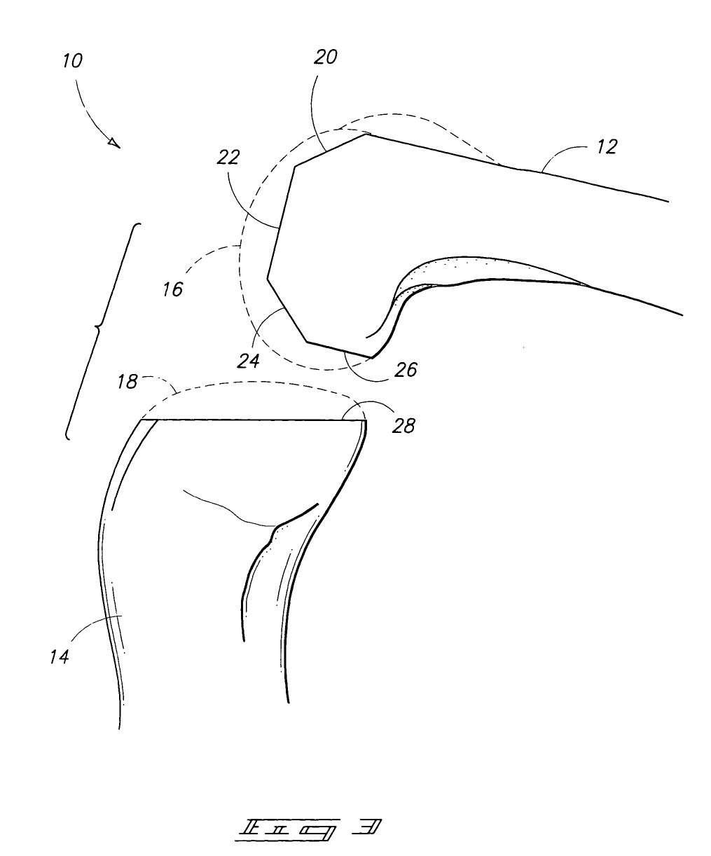

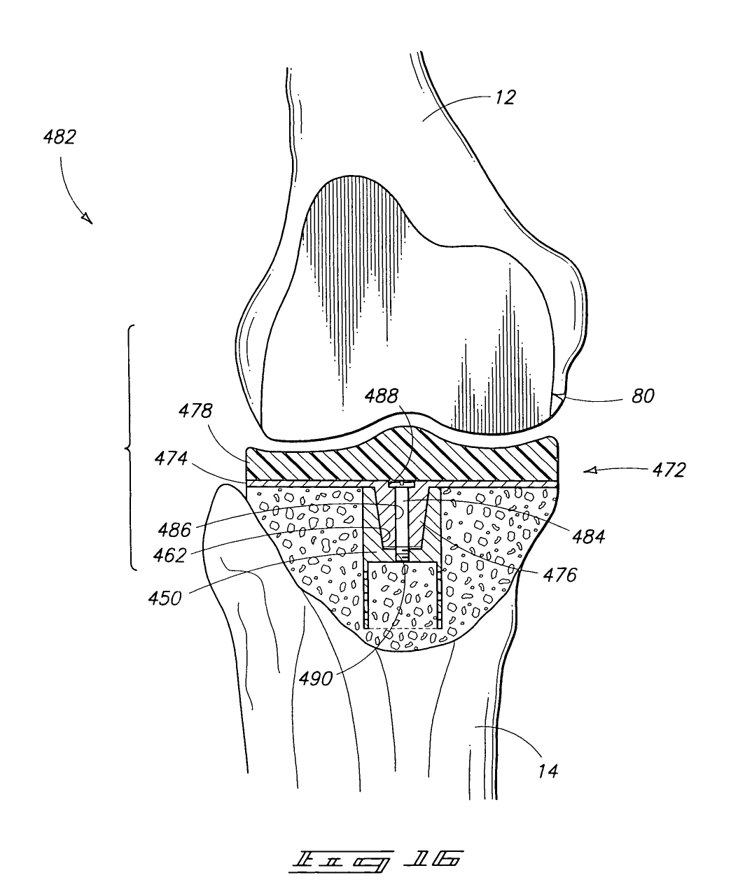

As discussed above with reference to the embodiments depicted in FIGS. 1-16, a cylindrical anchor has been presented in the form of a modular component used in a total knee replacement arthroplasty in order to provide bone fixation of a tibial baseplate. The apparatus and method for fixing the cylindrical anchor to a baseplate has been depicted using a Morse taper connection (via an interlocking pin and recess). Such a Morse taper construction has been used by a number of manufacturers making current knee arthroplasty systems, wherein a modular component uses a Morse taper within an armamentarium. However, it is understood that a Morse taper (pin and recess) forms but one apparatus and method for connecting a baseplate and a cylindrical anchor. However, other methods of connection are also possible, including the methods depicted below with reference to FIG. 17-29, as well as other apparatus and methods.

FIG. 17 depicts a fifth alternative embodiment for a knee joint prosthesis 582 having a further alternative tibial joint interface component 572 that differs from the component 472 in the embodiment of FIG. 16. More particularly, prosthesis 582 comprises a femoral joint interface component 80 provided on an end of a femur 12 and a tibial joint interface component 572 provided on an end of a tibia 14. Tibial joint interface component 572 includes a tibial baseplate 574 and an anchor 550 that differ over the respective components shown in the embodiment depicted in FIG. 16. Condyle interface component 578 is affixed atop tibial baseplate 574 in a manner similar to the respective components depicted in the embodiment of FIG. 16.

More particularly, tibial baseplate 574 includes a frustoconical aperture 588 configured to receive a tapered recessed head of a fastener 584 that threads into engagement within a threaded bore 590 of anchor 550. Unlike the embodiment depicted in FIG. 16, baseplate 574 does not include a Morse tapering pin and anchor 550 does not include a Morse tapering recess. Instead, recessed threaded fastener 584 provides the structural affixation between tibial baseplate 574 and anchor 550. Optionally, interlocking ribs and slots can be provided between baseplate 574 and anchor 550.

According to the embodiment depicted in FIG. 17, a fastener, such as a screw or bolt, is provided through a baseplate and into a cylindrical anchor in order to facilitate introduction of a baseplate onto the anchor. Such construction does not require separation of the femur from a tibial surface to allow vertical introduction of a male component on a Morse taper on the baseplate into a female receiver on the anchor. Accordingly, such construction requires less articulation and separation of the femur from the tibial surface during insertion. In the case for the screw or bolt that is used in the embodiment of FIG. 17, the baseplate can be slipped over the cylindrical anchor, providing just enough separation in order to allow the thickness of the baseplate to pass therethrough. Such a system could also be used with a relatively shorter anchor, such as a cylindrical or rectangular anchor in order to provide an anchorage for connection to the baseplate. Accordingly, a greater length of the anchor can be provided for bone fixtration by providing for more fenestrations therethrough.

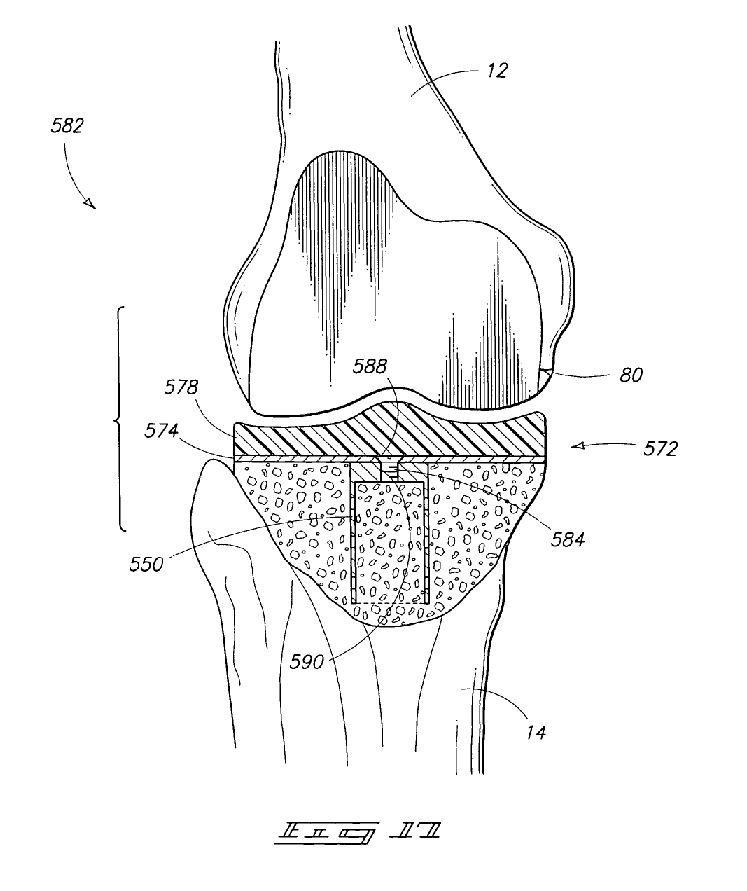

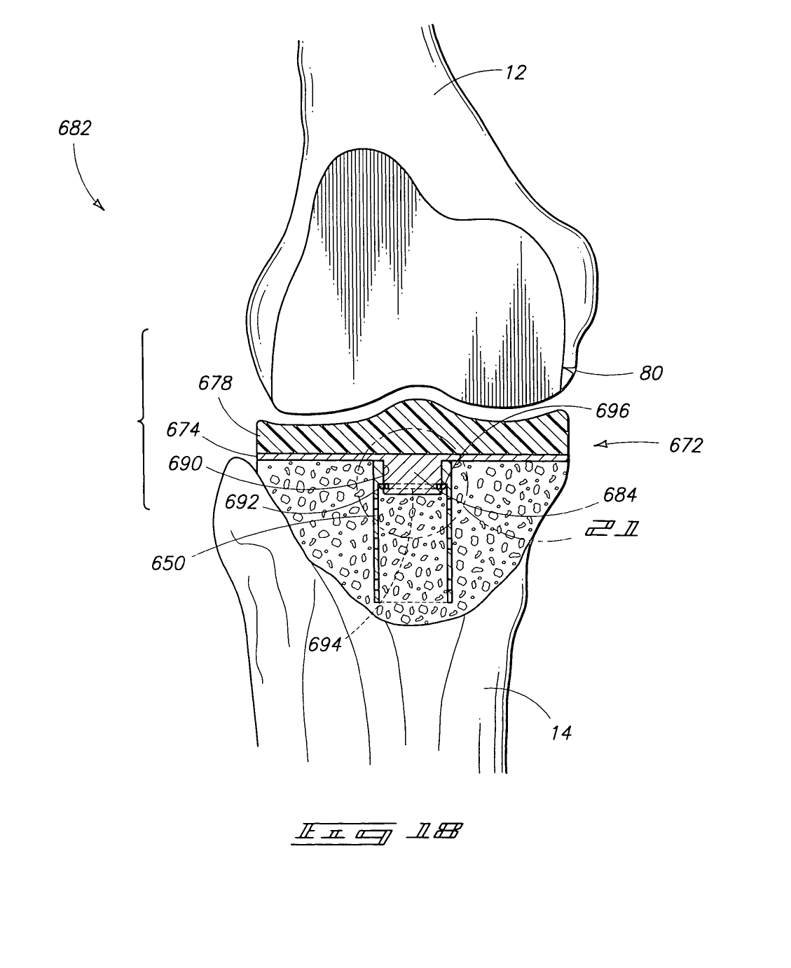

FIGS. 18-21 illustrate a sixth alternative construction for a knee joint prosthesis 682 having an even further alternative tibial joint interface component 672 that differs from component 572 (of FIG. 17). More particularly, prosthesis 682 includes a femoral joint interface component 80 provided on an end of femur 12 and a tibial joint interface component 672 provided on an end of tibia 14. Tibial joint interface component 672 includes a tibial baseplate 674 and an anchor, both of which differ over the respective components shown in the embodiment depicted in FIG. 17. A condial interface component 678 is affixed atop tibial baseplate 674 in a manner similar to the respective components depicted in the embodiment of FIG. 17. Further details of the construction and assembly between baseplate 674 and anchor 650 are provided below with reference to FIGS. 19-21.

More particularly, anchor 650 is inserted into a cylindrical bore similar to that depicted in FIGS. 5-6 within a tibia 14. Subsequently, a cylindrical pin 684 on tibial baseplate 674 is inserted within a complementary cylindrical bore 690 of anchor 650. A beveled, cylindrical retainer ring 692 is provided within a groove 694 on a distal end of post (or pin) 684, as shown in FIG. 19. Ring 692 is constructed from spring steel. Anchor 650 has a radial outwardly extending circumferential shelf 696 provided at a distal end of cylindrical bore 690. A leading surface bevel edge on retaining ring 692 circumferentially squeezes retaining ring 692 inwardly to enable passage of ring 692 through bore 690. Upon passage of retaining ring 692 through bore 690, ring 692 expands to seat against circumferential shelf 696, thereby locking pin 684 within bore 690. Accordingly, baseplate 674 is locked onto anchor 650.

In order to remove baseplate 674 from anchor 650, such as during a revision surgery, an arcuate aperture 695 is provided in pin 684 through which a tool (having a pair of inwardly compressible pins) can be received to compress radially inward lobes on retainer ring 692 in order to circumferentially compress ring 692 and extract pin 684 through cylindrical bore 690 of anchor 650. Accordingly, baseplate 674 can be removed from anchor 650 during a revision surgery.

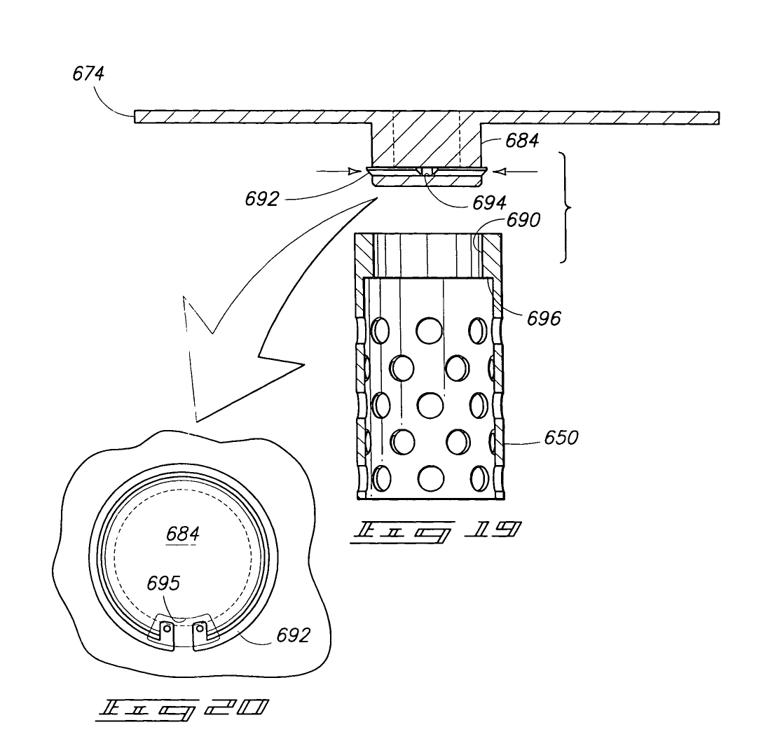

FIG. 21 shows in greater detail the position of retainer ring 692 locked in seated engagement with circumferential shoulder 696, with pin (or post) 684 received in complementary coaxial relation within bore 690. Circumferential groove 694 is provided in spaced relation relative to a bottom surface on baseplate 674 so that baseplate 674 is snugly received against a top edge of anchor 650 when retainer ring 692 snaps into engagement along circumferential shelf 696, thereby rigidly securing baseplate 674 atop anchor 650.

The embodiment depicted in FIGS. 18-21 consists of a ring lock type of connection, such as a retainer ring or a friction ring. The ring can be provided either on the post of the baseplate or on the receiving portion of the anchor. Correspondingly, a groove is provided on the opposite component configured to receive the ring. Such a construction typically would require significantly more femur-tibial separation in order to insert components of the knee joint into the space, such as is required when using a Morse taper pin and recess. Typically, it would also tend to occupy more of the internal length of the anchor in order to form a connection between the baseplate and the anchor, and provide less surface area for bone ingrowth through the fenestrations in the anchor, similar to the case for the Morse taper connection.

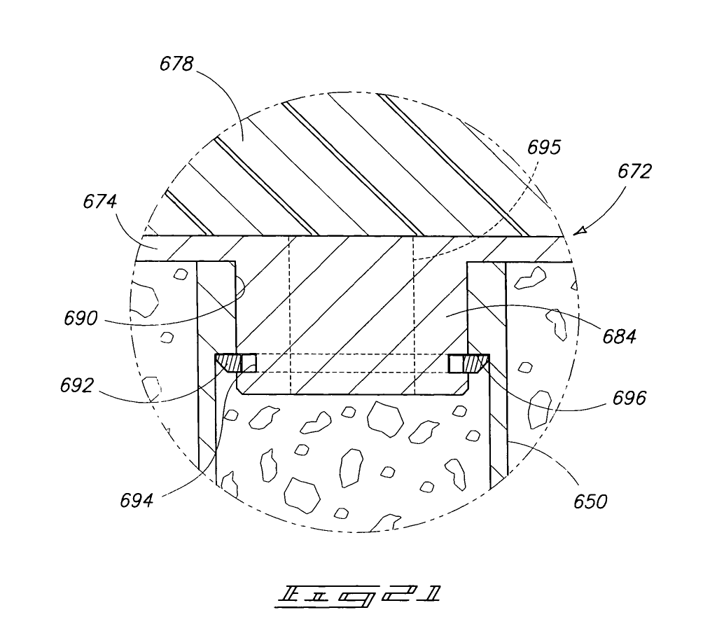

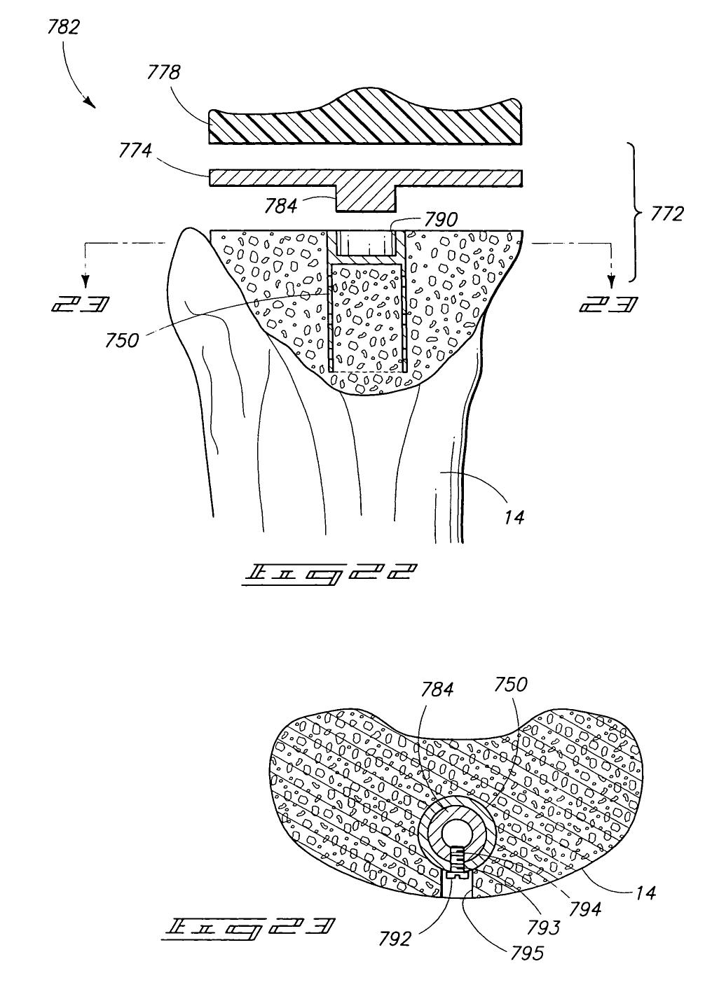

FIGS. 22-23 illustrate a seventh alternative embodiment for a knee joint prosthesis 782 omitting the joint interface component 80 (see FIG. 18). In addition to a joint interface component, prosthesis 782 includes a joint interface component 772 that is a further modification of those depicted in previous embodiments. More particularly, joint interface component 772 includes condial interface component 778 which is affixed onto a tibial baseplate 774 in a manner similar to that depicted in the previous embodiments, such as that depicted in FIG. 7. Joint interface component 772 also includes an anchor 750 that is received into a prepared tibia in a manner similar to the embodiments previously depicted.

A cylindrical post, or pin 784 depends from baseplate 774. The bottom end of post 784 is hollow, and an outer surface of post 784 is cylindrical and forms a complementary fit within a cylindrical bore 790 in a top portion of anchor 750.

As shown in FIG. 23, baseplate 774 is secured onto anchor 750, after anchor 750 has been implanted into tibia 14. More particularly, a screw 792 is inserted through a bore 793 provided in a wall portion of anchor 750. Screw 792 then threads into a complementary threaded bore 794 provided in post 784. By tightening screw 792 within the threaded bore 794, baseplate 774 and post 784 are rigidly affixed onto an upper end of anchor 750.

The embodiment depicted in FIGS. 22-23 utilizes a set screw that passes through an upper edge of the anchor and is further received into a central post or pin on the baseplate. Such as fastening mechanism requires that a surgical slot be placed in an anterior location of the tibia in order to allow insertion of the set screw. It is anticipated that this system will require some degree of additional surgical effort in order to align the anchor and baseplate to match alignment of the set screw holes. However, such a construction would be relatively easy to disengage in the event of a revision surgery, should removal of the baseplate be necessary at a later point in time.

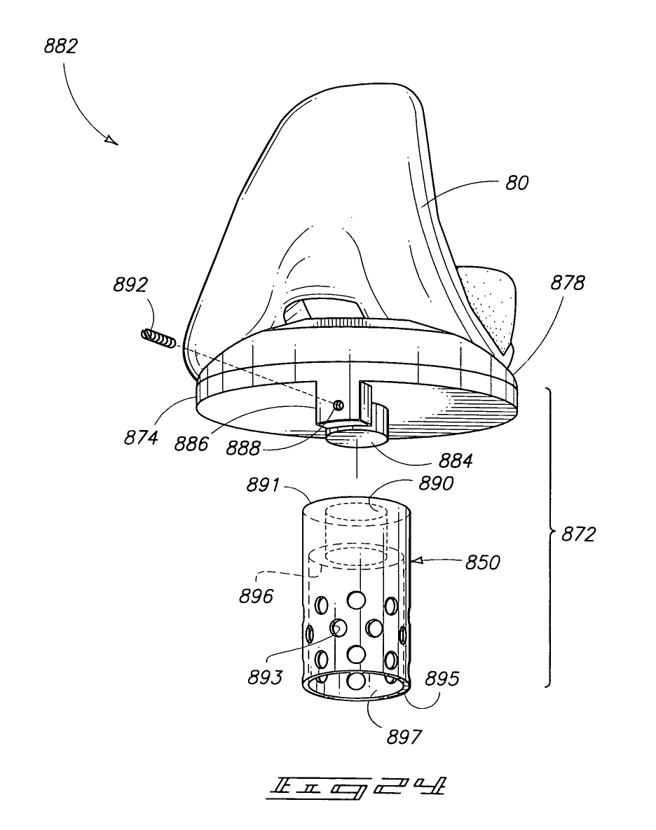

FIGS. 24-26 illustrate an eighth alternative embodiment for a knee joint prosthesis 882 having a joint interface component 80 and an alternatively constructed joint interface component 872. Joint interface component 872 includes an anchor 850 that is configured and surgically implanted in a manner similar to that shown for the previous embodiments.

Joint interface component 872 includes a tibial baseplate 874 that includes a downwardly depending cylindrical post 884 and a downwardly depending finger 886. Finger 886 includes a threaded bore 888 into which a threaded set screw 892 is received to clamp baseplate 874 atop anchor 850. Post 884 is received within a complementary bore 890 atop anchor 850, after anchor 850 has been surgically implanted into a tibia. An inner bore 897 within anchor 850 terminates in a circumferential shelf 896 into which a plug of living bone is received during implantation. Fenestrations 893 in an outer circumferential wall 895 of implant 850 promote bone ingrowth and throughgrowth upon surgical implantation.

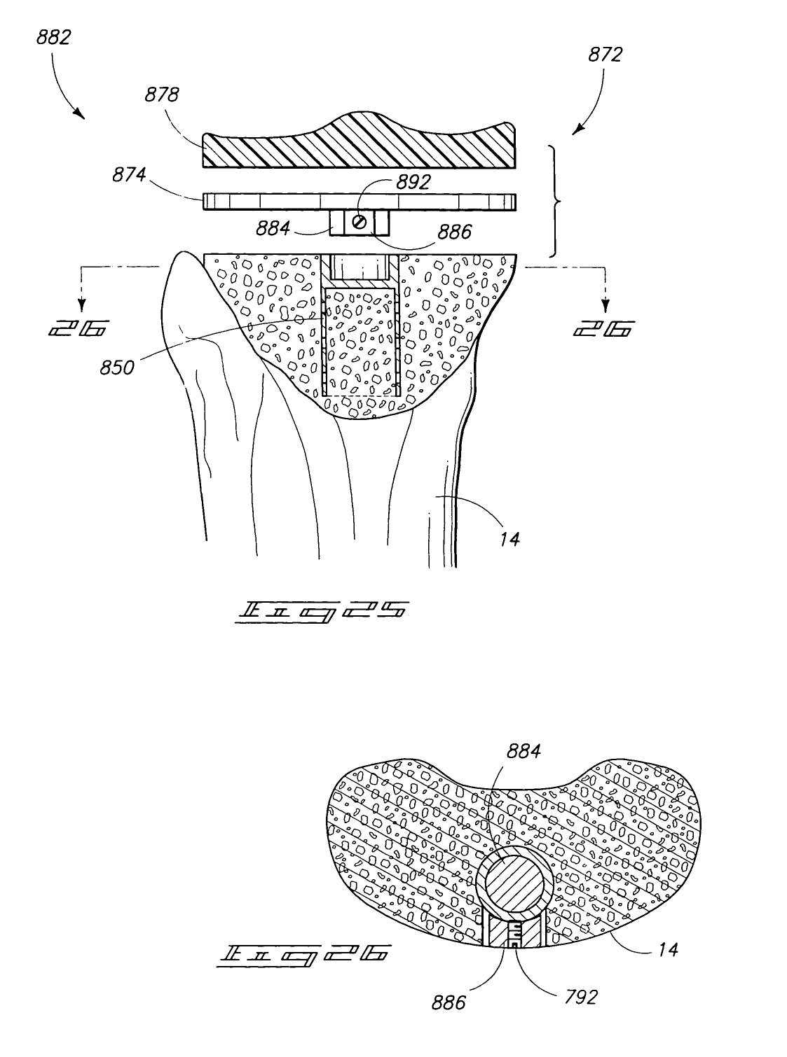

As shown in FIG. 25, baseplate 874 is shown in side view, whereas condial interface component 878 is shown in vertical sectional view along with a corresponding sectional view taken through tibia 14 and anchor 850. In this manner, the position of set screw 892 relative to finger 886 and post 884 can clearly be seen.

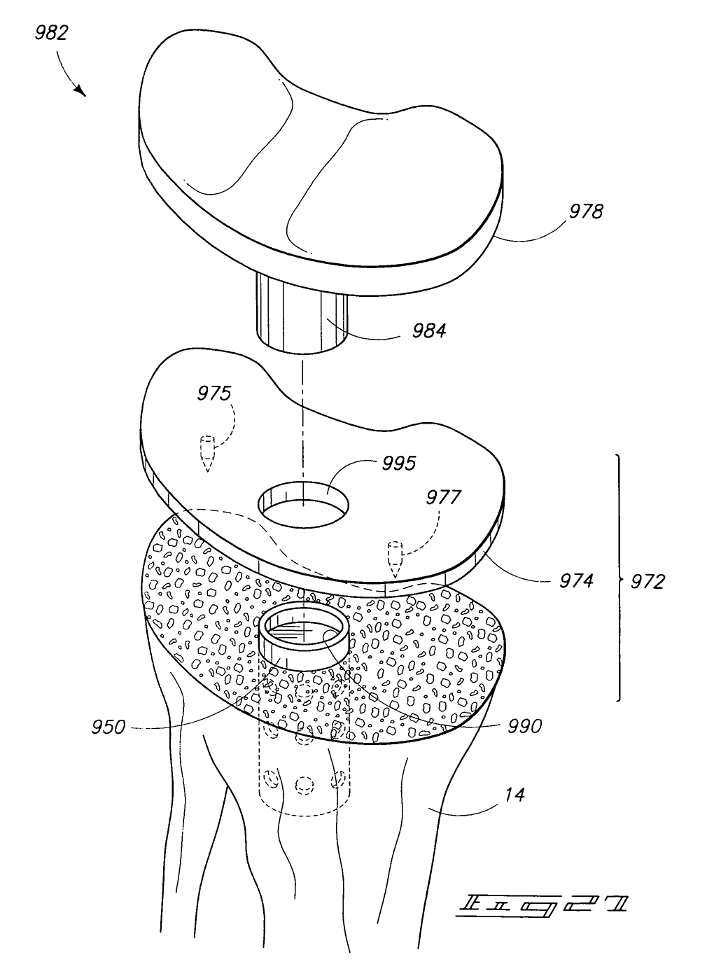

FIG. 26 further illustrates the placement of post 884 within an open cylindrical top portion 891 of anchor 850. Finger 886 is clearly shown positioned such that set screw 792 is tightened to lock post 884 and finger 886 about cylindrical portion 891. An aperture is provided in a front surface of tibia 14 to enable access to set screw 792 during surgical implantation.

According to the construction depicted in FIGS. 24-26, an alternative configuration is provided where a finger or projection is provided anterior to the post of the baseplate. A set screw enters through this finger or projection into the anchor wall. Such positioning of a set screw would require extra surgical alignment efforts in order to align the set screw. Additionally, a slot will need to be surgically provided in the anterior tibia. However, the slot would help a surgeon in ensuring alignment in an accurate proper orientation of the baseplate relative to the tibia, and would further prevent rotation of the baseplate, precluding the need for additional anti-rotation mechanisms in the construction.

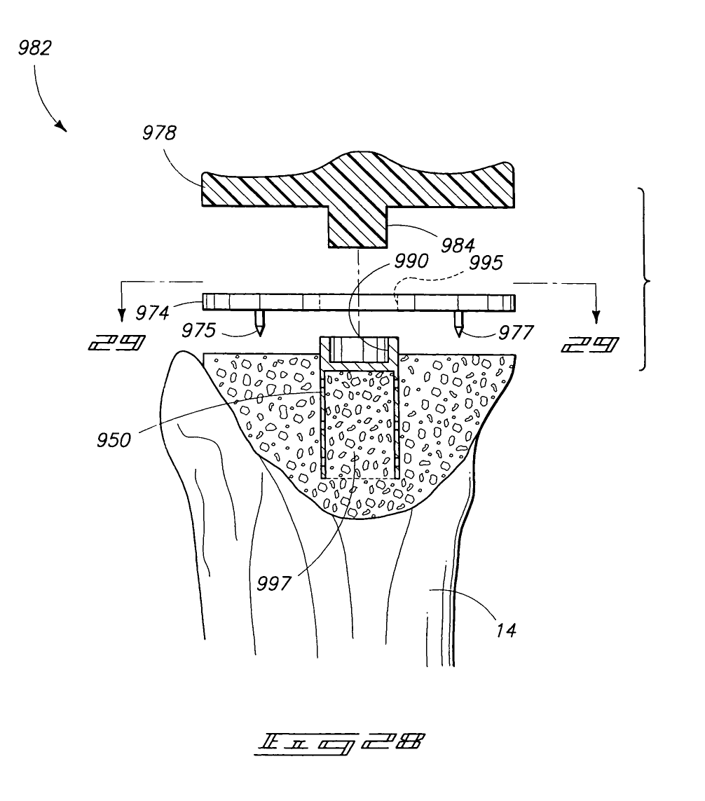

FIG. 27 illustrates a ninth alternative construction for a knee joint prosthesis 982. More particularly, prosthesis 982 includes an alternatively constructed condial interface component 978 as well as an alternatively constructed joint interface component 972. Condial interface component 978 is made from a low friction material such as a high density polyethelene (HDP) with a cylindrical stem portion 984 that enables condial interface 978 to rotate relative to joint interface component 972 and tibia 14. It is understood that a joint interface component, such as joint interface component 80 of FIG. 9 is also included in prosthesis 982, but has been omitted from the figure in order to focus on the novel alternative aspects of the present embodiment.

More particularly, joint interface component 972 includes a tibial baseplate 974 having a clearance bore 995 and a pair of downwardly depending studs 975 and 977 that are forcibly urged into porous bone that has been exposed in tibia 14 due to a surgical procedure. Clearance bore 995 is received about an outer diameter of anchor 950 which has been previously surgically implanted into tibia 14. A cylindrical end portion 990 of anchor 950 receives baseplate 974 via bore 995. Anchor pins 975 and 977 ensure that tibial baseplate 974 does not rotate relative to tibia 14. However, cylindrical post 984 on condial interface component 978 enables condial interface 978 to rotate atop tibial baseplate 974 to encourage alignment with a joint interface component on a femur, such as joint interface component 80 (see FIG. 6).

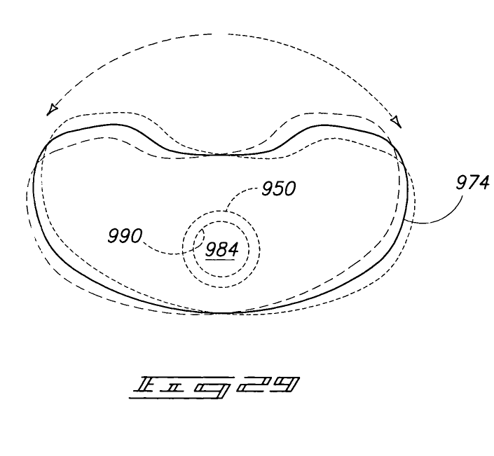

As shown in FIG. 28, tibial baseplate 974 is shown in side view, whereas condial interface component 978 and anchor 950 are shown in center line sectional view. Clearance bore 995 on baseplate 974 is received about an outer diameter of anchor 950, while anchor 950 extends upwardly from a top-most surface of tibia 14. Anchor pins 975 and 977 are forcibly urged downwardly into the bone of tibia 14, typically by forcibly urging or hammering faceplate 974 into flush engagement atop tibia 14. An inner bore 990 of implant 950 receives cylindrical post 984 of condial interface component 978. Cylindrical inner surface 994 provides a bearing surface in which post 984 is seated to enable condial interface component 978 to rotate into alignment with a complementary joint interface on a femur. Upon implantation, anchor 950 receives a living, contiguous bone projection 997 which forms bone ingrowth and bone throughgrowth to lock anchor 950 into tibia 14.

FIG. 29 further illustrates the self-aligning features provided by tibial baseplate 974 relative to anchor 950. More particularly, cylindrical post 984 of tibial baseplate 974 rotates within cylindrical bearing surface 980 of anchor 950, whereas anchor 950 is securely retained in rigid relation relative to a tibia.

According to the embodiment depicted in FIGS. 27-29, the anchor is mounted into a tibia so that it projects slightly above a top surface of the tibia, and the tibial baseplate is placed over this portion of the anchor. As an optional feature, component 978 can have a retainer ring or friction ring that locks within a complementary groove within cylindrical recess 990 of anchor 950. Further optionally, a set screw could be utilized through an aperture or slot in cylindrical surface 990 of anchor 950 that is affixed into a threaded bore within post 984 of component 978. Accordingly, component 978 could not separate from baseplate 974 and anchor 950. For the case where component 978 is made from a solid piece of polyethelene, post 984 would be configured to articulate directly within cylindrical recess 990 of anchor 950. Such a construction would provide a mobile bearing insert that can rotate and/or float between component 978 and anchor 950.

According to the embodiments depicted in FIGS. 1-9 above, examples are provided for various surgical apparatus and methods to accomplish connection of components in a total knee replacement surgery. The construction of an elongated anchor provides a component that can be used in both cemented and uncemented arthroplasty, as well as for fixating components to individual bones. The use of total knee arthroplasty has been addressed, but such an anchor system might also be applicable to arthroplasties involving underlying cancellous bone, such as hip and shoulder resurfacing, unicondylar knee arthroplasty, total ankle arthroplasty, and other surgical arthroplasties.

It is understood that the present techniques, apparatus, and methods can be provided on a number of other joint surfaces such as femoral condyles, a femoral head, a humeral head, as well as ankle joint surfaces. Such techniques eliminate the need to use bone cement when affixing the prosthesis to the bone.

In compliance with the statute, the invention has been described in language more or less specific as to structural and methodical features. It is to be understood, however, that the invention is not limited to the specific features shown and described, since the means herein disclosed comprise preferred forms of putting the invention into effect. The invention is, therefore, claimed in any of its forms or modifications within the proper scope of the appended claims appropriately interpreted in accordance with the doctrine of equivalents.

The invention claimed is:

1. A prosthesis anchor component, comprising:
a mounting stud having an elongated body with a tubular distal end and a proximal end, the tubular distal end including a tubular wall portion with an open leading end and a plurality of apertures extending through the tubular wall portion, and the proximal end having a mounting interface for securing a joint interface onto the stud.

2. The prosthesis anchor component of claim 1, wherein the stud comprises a fenestrated hollow metal cylinder with a cylindrical open leading end.

3. The prosthesis anchor component of claim 1, wherein the tubular distal end comprises a square cross-sectional end.

4. The prosthesis anchor component of claim 2, wherein the mounting interface comprises a Morse tapered pin.

5. The prosthesis anchor component of claim 1, wherein the tubular wall portion comprises a cylindrical wall.

6. The prosthesis anchor component of claim 1, wherein the apertures comprise cylindrical bores extending through the tubular wall portion.

7. The prosthesis anchor component of claim 1, wherein the open leading end communicates with a cylindrical bore in the tubular distal end, and the cylindrical bore terminates at a cylindrical end wall.

8. The prosthesis anchor component of claim 1, wherein a plurality of slots are provided in the proximal end of the elongated body.

9. A prosthesis anchor, comprising:
a mounting stem having a tubular leading end portion and a trailing end portion, the leading end portion having an open end communicating with a hollow interior portion and the trailing end portion having a mounting interface for securing a joint interface component onto the stud.

10. The prosthesis anchor of claim 9, wherein the mounting stem comprises a fenestrated hollow metal cylinder, and the open leading end and the hollow interior portion have an axially extending and substantially uniform interior surface configured to receive a vascularized pedicle of bone.

11. The prosthesis anchor of claim 10, wherein the interior surface of the hollow interior portion comprises a cylindrical bore.

12. The prosthesis anchor of claim 9, wherein the mounting stem comprises a cylinder having a hollow leading end.

13. The prosthesis anchor of claim 9, wherein the mounting stem has an outer surface with a rectangular cross-section.

14. The prosthesis anchor of claim 9, wherein the mounting interface comprises a tapered pin.

15. The prosthesis anchor of claim 14, wherein the tapered pin comprises a Morse taper.

16. A prosthesis, comprising:
a mounting anchor having a tubular leading end portion and a trailing end portion, the leading end portion having an open end communicating with a hollow interior portion and the trailing end portion having a first mounting interface; and
a joint interface component having a second mounting interface configured to mate in engagement with the first mounting interface to secure the joint interface component onto the mounting anchor.

17. The prosthesis of claim 16, wherein the mounting anchor comprises a mounting stem.

18. The prosthesis of claim 16, wherein the tubular leading end comprises a cylindrical wall having an open leading end communicating with a cylindrical inner surface.

19. The prosthesis of claim 16, wherein the joint interface comprises a base plate and the second mounting interface comprises a Morse tapering pin depending from the base plate.

20. The prosthesis of claim 19, wherein the joint interface further comprises a joint interface component configured to mate in articulating relation with a complementary joint interface.

21. The prosthesis of claim 19, wherein the joint interface further comprises at least two ribs extending between the base plate and the Morse tapering pin.

22. A method for affixing a joint interface component to a bone, comprising:
providing a mounting anchor having a fenestrated, tubular leading end portion and a trailing end portion with a female mounting interface and a joint interface component having a male mounting interface configured to mate with the first mounting interface;
preparing a receptacle within a bone proximate a resected bone joint interface having a tubular groove extending about a vascularized pedicle of bone;
inserting the mounting anchor into the receptacle with the tubular leading end received within the tubular groove; and
mating together the male mounting interface and the female mounting interface to engage together the mounting anchor and the joint interface.

23. The method of claim 22, further comprising, after mating together, fusing together the mounting anchor with the bone via fenestrations in the tubular leading end portion.

24. The method of claim 22, wherein the mounting anchor has a cylindrical leading end portion with a plurality of cylindrical fenestrations extending through a cylindrical wall of the cylindrical leading end portion.
